(19) United States
(12) Patent Application Publication
(10) Pub. No.: US 2023/0128120 A1
(43) Pub. Date: Apr. 27, 2023

(54) OMEGA MURICHOLIC ACID AS A PREGNANE X RECEPTOR LIGAND FOR TREATING HEPATO-INTESTINAL DISEASES

(71) Applicants: University of Washington, Seattle, WA (US); Albert Einstein College of Medicine, Bronx, NY (US)

(72) Inventors: Julia Yue Cui, Seattle, WA (US); Sridhar Mani, Bronx, NY (US); Moumita Dutta, Seattle, WA (US)

(73) Assignees: University of Washington, Seattle, WA (US); Albert Einstein College of Medicine, Bronx, NY (US)

(21) Appl. No.: 18/048,719

(22) Filed: Oct. 21, 2022

Related U.S. Application Data
(60) Provisional application No. 63/270,450, filed on Oct. 21, 2021.

(51) Int. Cl. A61K 31/575; A61K 35/74; A61K 9/00; A61P 1/16; C12P 33/00

(52) U.S. Cl. CPC A61K 31/575; A61K 9/0053; A61K 35/74; A61P 1/16; C12P 33/00

(57) ABSTRACT

Methods for activating pregnane X receptor (PXR) using co-muricholic acid thereby treating metabolic syndrome, obesity, inflammatory bowel disease, Crohn’s disease, and liver disease, and also thereby increasing CYP3A4 gene and/or protein expression. Also provided are related methods for activating PXR using β-muricholic acid and a bacterium capable of converting β-muricholic acid to co-muricholic acid.

### CROSS-REFERENCE TO RELATED APPLICATION

This application claims the benefit of U.S. Pat. Application No. 63/270,450, filed Oct. 21, 2021, expressly incorporated herein by reference in its entirety.

### STATEMENT OF GOVERNMENT LICENSE RIGHTS

This invention was made with government support under Grant No. R01 ES030197, awarded by the National Institutes of Health. The government has certain rights in the invention.

### BACKGROUND

Animal bile has been used successfully for centuries in traditional Asian medicine as was first documented in citations in Variorum of Shen Nong’s Herbal Classic (c. 492 CE), and deconvoluting the active component of bile remains an active area for modern allopathic research. For example, the bear bile was employed therapeutically for more than 2,500 years in China to treat jaundice, and its active bile acid (BA) ingredient ursodeoxycholic acid (UDCA) is now the only FDA-approved drug to treat primary biliary cholangitis. BAs are generated from cholesterol metabolism as primary BAs, which are further metabolized by intestinal bacteria to form secondary BAs. Novel microbial BAs are now being resolved; however, many of their functions and targets remain unknown.

Inflammatory bowel disease (IBD) is a significant public health issue. According to Centers for Disease Control and Prevention (CDC), there has been a large increase in the prevalence of IBD in United States from 1999 - 2021, impacting approximately 1.3% or 3 million adults in this country. IBD impacts all race and ethnicity groups, and with a higher increase in its prevalence rate in non-Hispanic Black adults in United States. Most people with IBD are diagnosed in their young adulthood (in their 20s and 30s), and adults with IBD have higher health care use than those without IBD leading to increased social and economic burdens. In addition, compared to adults without IBD, IBD patients have a much higher risk of developing other chronic health conditions including respiratory disease, cardiovascular disease, and cancer, making them more vulnerable during the COVID-19 pandemic. For example, a study found that ulcerative colitis patients are more likely to be hospitalized for COVID-19 compared to those without IBD. There are two major types of IBD, namely Crohn’s disease and ulcerative colitis, and both are chronic, immune-mediated diseases. IBD patients experience abnormal symptoms including diarrhea, abdominal pain, bloody stools and vomiting; among them Crohn’s disease affects most commonly distal ileum and colon and rarely upper part of gastrointestinal tract and rectum, whereas ulcerative colitis mainly affects colon.

The pathogenesis of IBD is complex and there is no single causative factor; it is thought that the host genome and environmental factors (diet, infection) play crucial roles. Diet plays an important role in regulating IBD. A study assessing dietary patterns among adults with IBD from the 2015 National Health Interview Survey found that IBD positively associated with lower dietary fiber intake, and women with IBD were more likely to have higher sugar intake. There is no known medical or surgical cure for IBD; treatment of IBD usually involves the use of anti-inflammatory drugs that can reduce the symptoms of the disease and help maintain its remission.

At the turn of the century, pregnane X receptor (PXR) was initially recognized as a critical xenobiotic-sensing transcription factor that modulates drug metabolism and disposition in liver and intestine where its expression is the highest. Many xenobiotics including drugs, environmental chemicals, and dietary factors can activate PXR, and this subsequently increases the transcription of numerous genes involved in xenobiotic biotransformation. PXR is highly expressed in the liver and intestine and can be activated by various drugs, nutraceuticals, dietary factors, and environmental chemicals, leading to drug-drug, drug-food, and drug-toxicant interactions. While being well recognized as a classic drug-receptor over the years, studies using PXR-null and humanized PXR transgenic mice have demonstrated that PXR also contributes to intermediary metabolism through worsening diet-induced obesity, hepatic steatosis, and hepatic inflammation. Recent studies also identified novel physiological functions of PXR in intermediary metabolism, including lipid metabolism and cell cycle.

In healthy individuals the microbiota aids in digestion, immunity, and metabolism. Metabolism of dietary factors and endogenously produced BAs provide important sensors and signals for the body. Emerging evidence in the literature has demonstrated that the onset of IBD involves a perturbation of the 100 trillion bacteria comprising the intestinal microbiota and the mucosal immune system. Dysbiosis has been observed in IBD patients, whereas fecal microbiota transplantation and probiotics have promising therapeutic potential in the management of IBD. However, the causative link between gut microbiota and IBD is not clearly understood. Furthermore, studies have not fully clarified the effectiveness, safety, and mechanism of this treatment.

Recently, increasing evidence in the literature demonstrates that PXR also has novel functions in inflammatory bowel diseases (IBD). In humans, case control studies of 422 IBD patients showed that genetic variation in the PXR gene is strongly associated with susceptibility to both Crohn’s disease and ulcerative colitis. Decreased expression of PXR has been reported in the colonic mucosa of IBD patients. Children with Crohn’s disease have decreased expression of the prototypical PXR target gene cytochrome P450 3A4 (CYP3A4), which is a major drug metabolizing enzyme and is the human ortholog of the mouse Cyp3a11, in the actively inflamed small intestine tissue. In addition to pro-inflammation, studies in IBD patients suggest that PXR-targeted detoxification enzyme depletion is an important event in the initiation and progression of ulcerative colitis. In colon biopsies from IBD patients, the PXR activator rifaximin reduces the expression of the pro-inflammatory cytokine interleukin 8 (IL-8). In human hepatocytes, the pro-inflammatory cytokine IL-6 decreased both the expression and activity of PXR, whereas PXR-null mouse lymphocytes had lower anti-inflammatory cytokine IL-10. In mice, pharmacological activation of PXR ameliorates dextran sulfate sodium (DSS) induced IBD-like phenotype via inhibition of NFκB-target gene expression in a PXR-dependent manner. In summary, the host PXR is a strong host factor for IBD and may serve as an important therapeutic target of the disease.

Obesity is a worldwide and major public health problem that results in 2.8 million deathseach year (World Health Organization (WHO). In 2016, the WHO stated that more than 1.9 billion adults were overweight, and 650 million people were obese. Nonalcoholic fatty liver disease (NAFLD) is the liver manifestation of obesity and metabolic syndrome and is a progressive disease that is usually accompanied by steatosis and inflammation, which may eventually evolve to NASH (non-alcoholic steatohepatitis) and liver cancer. NAFLD is one of the most prevalent liver diseases and affects approximately 30-40% of the US population and 25% of the world population. There are many contributing factors of an imbalance between energy intake and energy expenditure that lead to the pathogenesis of obesity and NAFLD. While modern western food eating habits and increased sedentary lifestyles are major contributory factors, researchers have found more evidence linking obesity with gut dysbiosis. High fat diet (HFD) induced activation of PXR worsens obesity as evidenced by protection from weight gain and liver steatosis in PXR-null mice. Therefore, masking the exogenous PXR activation through inverse agonist strategies may serve as a novel therapy for liver diseases.

Recent clinical studies revealed heightened the risk of comorbid liver diseases such as nonalcoholic fatty liver disease (NAFLD) and nonalcoholic steatohepatitis (NASH) in IBD patients and suggested that immune-mediated factors may be involved; however, the precise relationship and etiology remain unknown.

Despite the advances in the understanding and treatment of hepto-intestinal diseases, a need exists for improved therapeutic agents for the treatment of hepato-intestinal diseases. The present disclosure seeks to fulfill this need and provides further related advantages.

### SUMMARY

In one aspect, the disclosure provides a method of activating a pregnane X receptor (PXR) in a subject.

In other aspects, the present disclosure provides methods of treating (a) metabolic syndrome, (b) obesity, (c) inflammatory bowel disease, (d) Crohn’s disease, or (e) liver disease in a subject.

In a further aspect, the present disclosure provides a method of increasing CYP3A4 gene and/or protein expression in a subject.

In certain embodiments of the above methods, the methods comprise administering to a subject in need thereof a therapeutically effective amount of ω-muricholic acid, or a pharmaceutically acceptable salt thereof.

In other embodiments of the above methods, ω-muricholic acid is generated in vivo from β-muricholic acid, or a pharmaceutically acceptable salt thereof, and a bacterium capable of converting β-muricholic acid, or a pharmaceutically acceptable salt thereof, to ω-muricholic acid, or a pharmaceutically acceptable saltthereof (e.g., a therapeutically effective amount of β-muricholic acid, or a pharmaceutically acceptable salt thereof, and a bacterium capable of converting β-muricholic acid, or a pharmaceutically acceptable salt thereof, to ω-muricholic acid, or a pharmaceutically acceptable salt thereof).

In further embodiments of the above methods, the methods further comprise administering to the subject β-muricholic acid, or a pharmaceutically acceptable salt thereof, concurrently with the ω-muricholic acid, or a pharmaceutically acceptable salt thereof. In certain of these embodiments, the method further comprises administering to the subject a bacterium capable of converting β-muricholic acid, or a pharmaceutically acceptable salt thereof, to ω-muricholic acid, or a pharmaceutically acceptable salt thereof.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a schematic illustration of bile acid (BA) metabolism as a joint effort of the liver and intestinal bacteria. Mouse specific BAs and the 6-isomerase activities of the microbes are highlighted.

FIG. 2A illustrates prototypical PXR-target gene Cyp3a11 protein expression in livers of age-matched conventional (CV, with normal microbiome) and germ free (GF) male mice at 7 developmental ages. The Cyp3a11 protein isoform was quantified using LC-MS and normalized to the total protein levels in microsomes as described in Li, C. Y. et al. Novel Interactions between Gut Microbiome and Host Drug-Processing Genes Modify the Hepatic Metabolism of the Environmental Chemicals Polybrominated Diphenyl Ethers. Drug Metab Dispos 45, 1197-1214, doi:10.1124/dmd.117.077024 (2017). Asterisks (*) represent statistically significant difference as compared to CV mice of the same age (student’s t-test, p < 0.05).

FIG. 2B illustrates prototypical PXR-target genes Cyp3a11 and Cyp3a41a expression in liver, and Cyp3a11 mRNA expression ileum of GF and ex-GF mice inoculated with human fecal microbiome from healthy pediatric donors of various developmental ages. Inclusion criteria: no current use of antibiotics or probiotics, no known liver or gastro-intestinal diseases; both genders, and any ethnicity. A total of 60 human subjects were recruited; fresh feces were immediately frozen and divided into four age ranges (1-week to about 6 months; 7-24 months; 3-5 years; and 5-12 years). One gram of human feces was diluted in 10 mL reduced sterile PBS under anaerobic conditions. At 1-month of age, GF mice were inoculated with one of the four age-configurations of the human microbiome via a single oral gavage of 100 µL of the fecal suspension. Control GF mice received sterile PBS (vehicle). Various bio-compartments were collected 1-monthlater for further analysis (n = 5 per group). This method has been shown to preserve the core features of the human microbiome from the donors in exGF mice (see Turnbaugh, P. J. et al. The effect of diet on the human gut microbiome: a metagenomic analysis in humanized gnotobiotic mice. Sci Transl Med 1, 6ra14, doi:10.1126/scitranslmed.3000322 (2009); and Selwyn, F. P., Csanaky, I. L., Zhang, Y. & Klaassen, C. D. Importance of Large Intestine in Regulating Bile Acids and Glucagon-Like Peptide-1 in Germ-Free Mice. Drug Metab Dispos 43, 1544-1556, doi:10.1124/dmd.115.065276(2015). The Cyp3a gene expression was quantified using RT-qPCR and normalized to the house keeping gene Gapdh. The letters (a, b, c) represent different post hoc groups, and bars with non-overlapping labels with the controls are considered statistically significant (one-way ANOVA followed by Ducan’s post hoc test, p < 0.05).

FIG. 3A shows relative percentages of various primary and secondary BAs in their taurine (T) conjugated or unconjugated forms in serum and large intestinal content of 2-months old adult CV and GF male mice (note: T-ωMCA not included in the UPLC- MS/MS panel).

FIG. 3B shows levels of ωMCA and HDCA in liver, large intestinal content, and serum of GF and human fecal microbiome inoculated exGF mice. Average values each BA from 5 biological replicates per group are used as input of the pie plots. Their taurine (T) conjugated forms are also shown if they were above the detection limit. BAs were quantified using UPLC-MS/MS. The procedures of inoculation of GF mice with human fecal microbiome are described in FIG. 2B legend. The letters (a, b, c) represent different post hoc groups, and bars with non-overlapping labels with the controls are considered statistically significant (one-way ANOVA followed by Duncan’s post hoc test, p < 0.05).

FIG. 4A shows Pearson’s correlation analysis between differentially regulated BAs (p < 0.05) (out of 20 major BAs present in mice from the UPLC-MS/MS panel) and the PXR-targeted Cyp3a genes in ileum and liver from the humanized exGF mouse study (n=5 per group) as illustrated in detail in FIG. 1B legend. Red represents positive Pearson’s correlation coefficient r value indicating positive associations; blue represents negative Pearson’s r value indicating negative associations.

FIG. 4B shows luciferase assays for mouse (m) PXR and human (h) PXR activation by their prototypical ligands (PCN for mPXR, and rifampicin [Rif] for hPXR), as well as by ωMCA of various concentrations. Data are expressed as average of relative luciferase units among the technical triplicates ± S.E. Asterisks (*) represent statistically significant difference as compared to the vehicle group (one-way ANOVA followed by Duncan’s post hoc test, p < 0.05).

FIG. 4C compares Cyp3a gene expression in livers of GF PXR-null mice.

FIG. 5A presents Pearson’s correlation analysis between differentially regulated BAs (p < 0.05) (out of 20 major BAs present in mice from the UPLC-MS/MS panel) and differentially regulated taxa from the large intestinal content of the humanized exGF mouse study (n=5 per group) as illustrated in detail in FIG. 2B legend. The gut microbiome was sequenced using 16S rDNA sequencing for the compositional changes (hypervariable V4 region, 250 paired-end, 0.1 million reads per sample)) using methods as described in Gomez, M. V. et al. Early Life Exposure to Environmental Contaminants (BDE- 47, TBBPA, and BPS) Produced Persistent Alterations in Fecal Microbiome in Adult Male Mice. Toxicol Sci 179, 14-30, doi:10.1093/toxsci/kfaa161 (2021); Cheng, S. L. et al. Gut Microbiota Modulates Interactions Between Polychlorinated Biphenyls and Bile Acid Homeostasis. Toxicol Sci 166, 269-287, doi:10.1093/toxsci/kfy208 (2018); Li, C. Y. et al. PBDEs Altered Gut Microbiome and Bile Acid Homeostasis in Male C57BL/6 Mice. Drug Metab Dispos 46, 1226-1240, doi:10.1124/dmd.118.081547 (2018); Lim, J. J. et al. Neonatal exposure to BPA, BDE-99, and PCB produces persistent changes in hepatic transcriptome associated with gut dysbiosis in adult mouse livers. Toxicol Sci, doi:10.1093/toxsci/kfab104 (2021). Deep metagenomic shotgun sequencing (20 million reads per sample) was performed using the same batch of samples for improved resolution of the species information of the taxa.

FIG. 5B presents Pearson’s correlation analysis between differentially regulated taxa from the large intestinal content and the PXR-targeted Cyp3a genes in ileum and liver from the large intestinal content of the humanized exGF mouse study. In both FIGS. 5A and 5B (positive Pearson’s correlation coefficient r values indicating positive associations; negative Pearson’s r values indicating negative associations).

FIG. 6 is a table summarizing microbes from inoculated human feces that positively associated with ωMCA in exGF mice.

FIG. 7 compares ωMCA production (in its taurine-conjugated form which is the majority its presence in liver circulation) in mouse intestine of ex-germ-free mice inoculated with live A. muciniphila or A. muciniphila+ L. acidiphillus (10⁹ CFU, single oral gavage, n = 3-5 per group). Samples were collected 1 month after inoculation. Asterisk: statistically significant difference as compared to vehicle (sterile phosphate buffered saline) treated group of the same sex; pound: statistically significant sex difference as compared to the same treatment group (ANOVA followed by Duncan’s post hoc test, p < 0.05).

FIGS. 8A and 8B compare DCA (8A) and LCA (8B) production after isolated cecum from germ free mice was infused with their corresponding precursors (cholic acid and chenodeoxycholic acid, respectively). Akk: A. muciniphila; Cs: clostridium scindens (positive control known to produce DCA and LCA). Asterisks: statistically significant difference as compared to vehicle (sterile phosphate buffered saline) treated group; pounds: statistically significant sex difference as compared to the A. muciniphila treated group (ANOVA followed by Duncan’s post hoc test, p < 0.05).

FIG. 9 illustrates the difference in binding affinity of omega muricholic acid (ωMCA) and murideoxycholic acid (MDCA). MDCA showed no binding affinity towards PXR in experimental settings. Reporter assay was performed using LS intestinal cells transfected with human PXR + reporter. Incubation time was 24 h and a dose-response curve was plotted. Results were confirmed using four different cells passages.

### DETAILED DESCRIPTION

The present disclosure provides methods for activating pregnane X receptor (PXR) using ω-muricholic acid thereby treating metabolic syndrome, obesity, inflammatory bowel disease, Crohn’s disease, and liver disease, and also thereby increasing CYP3A4 gene and/or protein expression. Also provided are related methods for activating pregnane X receptor (PXR) using β-muricholic acid and a bacterium capable of converting β-muricholic acid to ω-muricholic acid.

In one aspect, the disclosure provides a method of activating a pregnane X receptor (PXR) in a subject.

In certain embodiments, the method comprises administering to a subject in need thereof a therapeutically effective amount of ω-muricholic acid, or a pharmaceutically acceptable salt thereof.

In other embodiments, ω-muricholic acid is generated in vivo from β-muricholic acid, or a pharmaceutically acceptable salt thereof, and a bacterium capable of converting β-muricholic acid, or a pharmaceutically acceptable salt thereof, to ω-muricholic acid, or a pharmaceutically acceptable salt thereof (e.g., a therapeutically effective amount of β-muricholic acid, or a pharmaceutically acceptable salt thereof, and a bacterium capable of converting β-muricholic acid, or a pharmaceutically acceptable salt thereof, to ω-muricholic acid, or a pharmaceutically acceptable salt thereof). In certain of these embodiments, the bacterium is selected from the group consisting of Akkermansia muciniphila, Parabacteroides distasonis, Faecalibacterium prausnitzii, Ruminococcus bromii, Ruminococcus callidus, Ruminococcus lactaris, Ruminoccus gnavus, Ruminococcus torques, and Coprobacillus sp. Strain D6, or a combination thereof.

In certain embodiments of the above methods, the method further comprises administering to the subject β-muricholic acid, or a pharmaceutically acceptable salt thereof, concurrently with the ω-muricholic acid, or a pharmaceutically acceptable salt thereof. In certain of these embodiments, the method further comprises administering to the subject a bacterium capable of converting β-muricholic acid, or a pharmaceutically acceptable salt thereof, to ω-muricholic acid, or a pharmaceutically acceptable salt thereof. In certain of these embodiments, the bacterium is selected from the group consisting of Akkermansia muciniphila, Parabacteroides distasonis, Faecalibacterium prausnitzii, Ruminococcus bromii, Ruminococcus callidus, Ruminococcus lactaris, Ruminoccus gnavus, Ruminococcus torques, and Coprobacillus sp. Strain D6, and combinations thereof.

In certain of the above methods, ω-muricholic acid, or a pharmaceutically acceptable salt thereof, is an inverse agonist of PXR.

In the above methods, activating PXR comprises contacting PXR with ω-muricholic acid, or a pharmaceutically acceptable salt thereof. In certain of these embodiments, PXR is selectively activated in the intestines of the subject.

In certain of the above methods, activation of PXR decreases inflammation in the intestines of the subject.

In certain of the above methods, administration comprises oral or rectal administration.

In certain of the above methods, ω-muricholic acid, or a pharmaceutically acceptable salt thereof, is administered at a ω-muricholic acid-equivalent dose compatible with the activation profile of PXR in the intestines and/or other organs.

In certain of the above methods, ω-muricholic acid, or a pharmaceutically acceptable salt thereof, comprises a dosage form selected from a solid dosage form, a liquid dosage form, or a suspension.

In certain of the above methods, the subject (e.g., human) has a condition selected from metabolic syndrome, obesity, inflammatory bowel disease, Crohn’s disease, liver disease (e.g., nonalcoholic fatty liver disease, nonalcoholic steatohepatitis), or a combination thereof.

In other aspects, the present disclosure provides methods of treating (a) metabolic syndrome, (b) obesity, (c) inflammatory bowel disease, (d) Crohn’s disease, or (e) liver disease in a subject.

In certain embodiments of these methods, treating the specified diseases and conditions comprises administering to a subject in need thereof a therapeutically effective amount of ω-muricholic acid, or a pharmaceutically acceptable salt thereof.

In other embodiments, ω-muricholic acid is generated in vivo from β-muricholic acid, or a pharmaceutically acceptable salt thereof, and a bacterium capable of converting β-muricholic acid, or a pharmaceutically acceptable salt thereof, to ω-muricholic acid, or a pharmaceutically acceptable salt thereof (e.g., a therapeutically effective amount of β-muricholic acid, or a pharmaceutically acceptable salt thereof, and a bacterium capable of converting β-muricholic acid, or a pharmaceutically acceptable salt thereof, to ω-muricholic acid, or a pharmaceutically acceptable salt thereof). In certain of these embodiments, the bacterium is selected from the group consisting of Akkermansia muciniphila, Parabacteroides distasonis, Faecalibacterium prausnitzii, Ruminococcus bromii, Ruminococcus callidus, Ruminococcus lactaris, Ruminoccus gnavus, Ruminococcus torques, and Coprobacillus sp. Strain D6, or a combination thereof.

In certain embodiments of the above methods, the method further comprises administering to the subject β-muricholic acid, or a pharmaceutically acceptable salt thereof, concurrently with the ω-muricholic acid, or a pharmaceutically acceptable salt thereof. In certain of these embodiments, the method further comprises administering to the subject a bacterium capable of converting β-muricholic acid, or a pharmaceutically acceptable salt thereof, to ω-muricholic acid, or a pharmaceutically acceptable salt thereof. In certain of these embodiments, the bacterium is selected from the group consisting of Akkermansia muciniphila, Parabacteroides distasonis, Faecalibacterium prausnitzii, Ruminococcus bromii, Ruminococcus callidus, Ruminococcus lactaris, Ruminoccus gnavus, Ruminococcus torques, and Coprobacillus sp. Strain D6, and combinations thereof.

In certain embodiments, treating the specified diseases and conditions further comprises treating a comorbid liver disease in the subject. In certain of these embodiments, the comorbid liver disease is selected from nonalcoholic fatty liver disease, nonalcoholic steatohepatitis, or a combination thereof.

In certain embodiments, treating liver disease comprises treating nonalcoholic fatty liver disease, nonalcoholic steatohepatitis, or both.

In a further aspect, the present disclosure provides a method of increasing CYP3A4 gene and/or protein expression in a subject.

In certain embodiments, the method comprises administering to a subject in need thereof a therapeutically effective amount of ω-muricholic acid, or a pharmaceutically acceptable salt thereof.

In other embodiments, ω-muricholic acid is generated in vivo from β-muricholic acid, or a pharmaceutically acceptable salt thereof, and a bacterium capable of converting β-muricholic acid, or a pharmaceutically acceptable salt thereof, to ω-muricholic acid, or a pharmaceutically acceptable salt thereof (e.g., a therapeutically effective amount of P-muricholic acid, or a pharmaceutically acceptable salt thereof, and a bacterium capable of converting β-muricholic acid, or a pharmaceutically acceptable salt thereof, to ω-muricholic acid, or a pharmaceutically acceptable salt thereof). In certain of these embodiments, the bacterium is selected from the group consisting of Akkermansia muciniphila, Parabacteroides distasonis, Faecalibacterium prausnitzii, Ruminococcus bromii, Ruminococcus callidus, Ruminococcus lactaris, Ruminoccus gnavus, Ruminococcus torques, and Coprobacillus sp. Strain D6, or a combination thereof.

In certain embodiments of the above methods, the method further comprises administering to the subject β-muricholic acid, or a pharmaceutically acceptable salt thereof, concurrently with the ω-muricholic acid, or a pharmaceutically acceptable salt thereof. In certain of these embodiments, the method further comprises administering to the subject a bacterium capable of converting β-muricholic acid, or a pharmaceutically acceptable salt thereof, to ω-muricholic acid, or a pharmaceutically acceptable salt thereof. In certain of these embodiments, the bacterium is selected from the group consisting of Akkermansia muciniphila, Parabacteroides distasonis, Faecalibacterium prausnitzii, Ruminococcus bromii, Ruminococcus callidus, Ruminococcus lactaris, Ruminoccus gnavus, Ruminococcus torques, and Coprobacillus sp. Strain D6, and combinations thereof.

In certain embodiments of the above methods, the PXR is human PXR.

In certain embodiments of the above methods, the subject is a human.

Omega muricholic acid (ωMCA), an abundant secondary BA in specific to mouse bile, has been discovered as a novel ligand of the major xenobiotic-sensing nuclear receptor pregnane X receptor (PXR). Livers of germ free (GF) mice have been demonstrated to exhibit a marked decrease in the hepatic and intestinal expression of the prototypical PXR-target gene cytochrome P450 (Cyp) 3a11, the human ortholog of which (CYP3A4) is responsible for metabolizing over 50% of the prescribed drugs in the market. Inoculation of germ free (GF) mice with human microbiome completely rescued the constitutive expression of Cyp3a11 in a PXR-dependent manner and was associated with a marked increase in ωMCA, as well as a reduction of its primary BA substrate beta muricholic acid (βMCA). Unlike the PXR-activating lithocholic acid (LCA), a well characterized secondary BA that can only activate PXR at toxic concentrations and its endogenous levels are well below the concentrations required to activate PXR, luciferase assays showed that ωMCA activates PXR of both mouse and human origins under physiological estimated concentrations. Activation of PXR in intestines abrogates intestinal inflammation and improves inflammatory bowel disease (IBD) in animal models. Strategies to limit locoregional activation of PXR in the intestines limit unnecessary systemic PXR activation, resulting in adverse drug interactions.

Recent clinical studies revealed heightened the risk of comorbid liver diseases such as nonalcoholic fatty liver disease (NAFLD) and nonalcoholic steatohepatitis (NASH) in IBD patients and suggested that immune-mediated factors may be involved; however, the precise relationship and etiology remain unknown (Digestive Disease Week 2019; https://consultqd.clevelandclinic.org/studies-reveal-heightened-liver-disease-rates-in-ibd-patients/). Hepatic ωMCA was found to decrease in a murine model of nonalcoholic steatohepatitis (NASH). PXR activation by high fat diet has shown to be detrimental to obesity and the development of NAFLD and NASH. Therefore, restoring ωMCA may serve as an inverse agonist to block the exogenous ligand-mediated activation of PXR, thereby serving a novel therapeutic strategy against NAFLD and NASH. Considering that the precursor of ωMCA - βMCA has already demonstrated promising therapeutic potential for metabolic disorders through antagonizing the host farnesoid X receptor (FXR), and obesity is prevalent among IBD patients (15-40%), a combination of βMCA + probiotics with ωMCA-producing capacity will bring even greater therapeutic values in patients with multiple complex diseases (e.g., IBD + NASH) through a “FXR ➔ PXR” 2-hit strategy.

The biochemical and molecular interactions of ωMCA on PXR, identification of additional novel microbes and microbial interactions in ωMCA production from its precursor βMCA, and testing the effect of ωMCA in several murine models of IBD and NASH, are described herein.

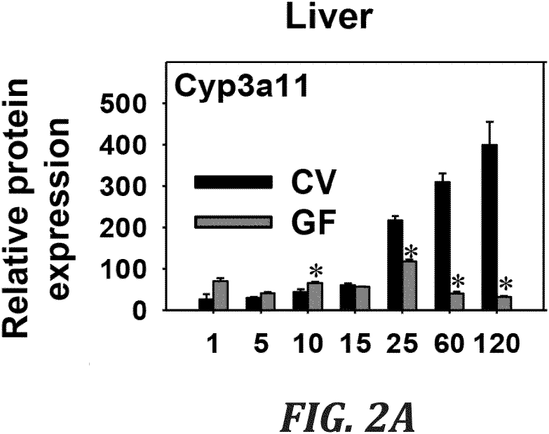

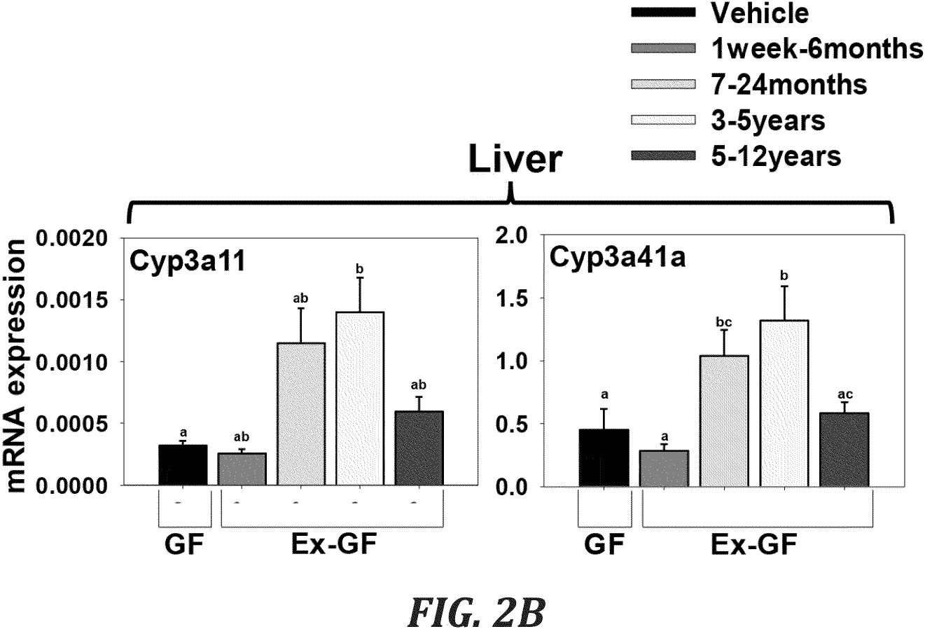

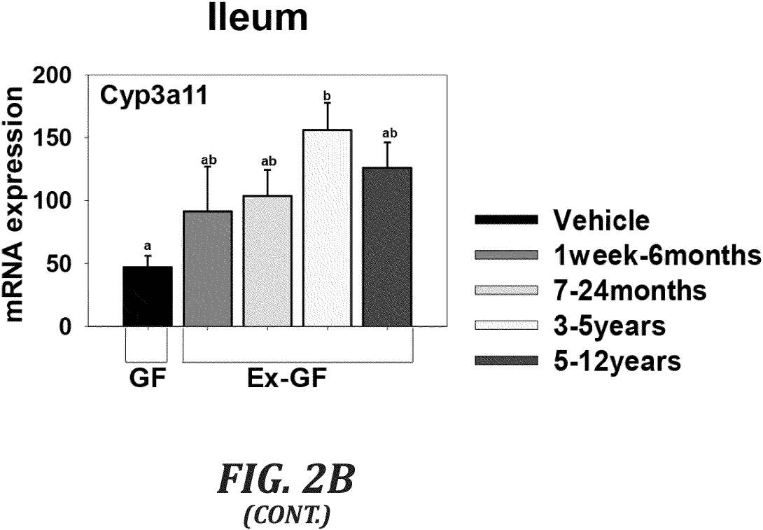

Gut microbiome is necessary in maintaining the constitutive expression of hepatic and intestinal prototypical PXR-target genes (Cyp3a). Using the Cyp3a genes as a read-out of PXR-signaling, LC-MS based quantitative protein analysis of the Cyp3a11 gene isoform in livers of control CV and GF male mice at various developmental ages were conducted (FIG. 2A). There was age-dependent decrease in Cyp3a11 protein expression in the absence of the gut microbiota with the greatest downregulation observed in 120-days adult age. Inoculating the GF mice with human fecal microbiome from healthy donors of various age ranges restored the mRNA expression of Cyp3a11 and Cyp3a41a (another PXR-target gene) in liver in a human donor’s age-specific manner, with the significant increase in the Cyp3a mRNAs observed in GF mice inoculated with the feces from 3 to about 5 years-old donors. The trend of the Cyp3a mRNA increase was also observed in livers of exGF mice inoculated with the feces from 7 to about 24 months old donors and 5 to about 12-year-old donors (FIG. 2B). In ileum, human fecal microbiome transplant also restored the Cyp3a11 mRNA expression, with the highest mRNA levels observed in ileum of exGF mice inoculated with the feces from 3 to about 5-year-old donors, and a trend of mRNA increase in the other recipient groups as well (FIG. 2B). In summary, the presence of the gut microbiome is necessary and sufficient in maintaining the constitutive PXR-signaling in liver and intestine as evidenced by the expression of its prototypical target genes Cyp3a in GF and exGF mice receiving fecal microbiome transplant from human feces of age-specific configurations.

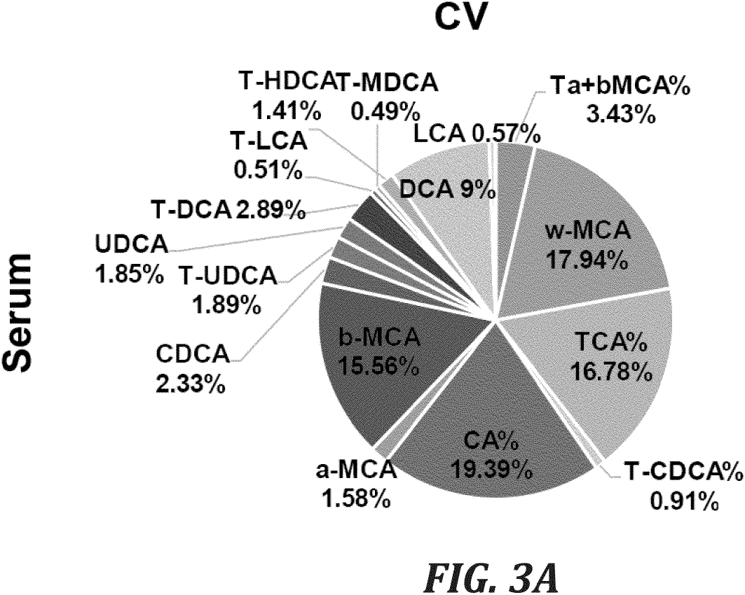

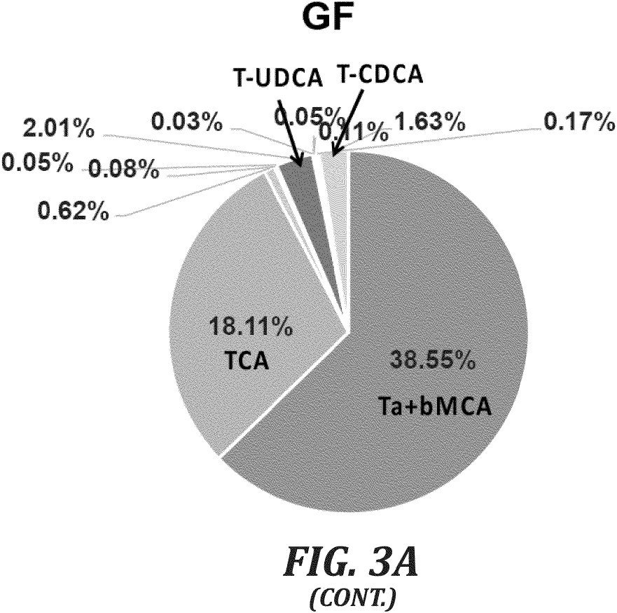

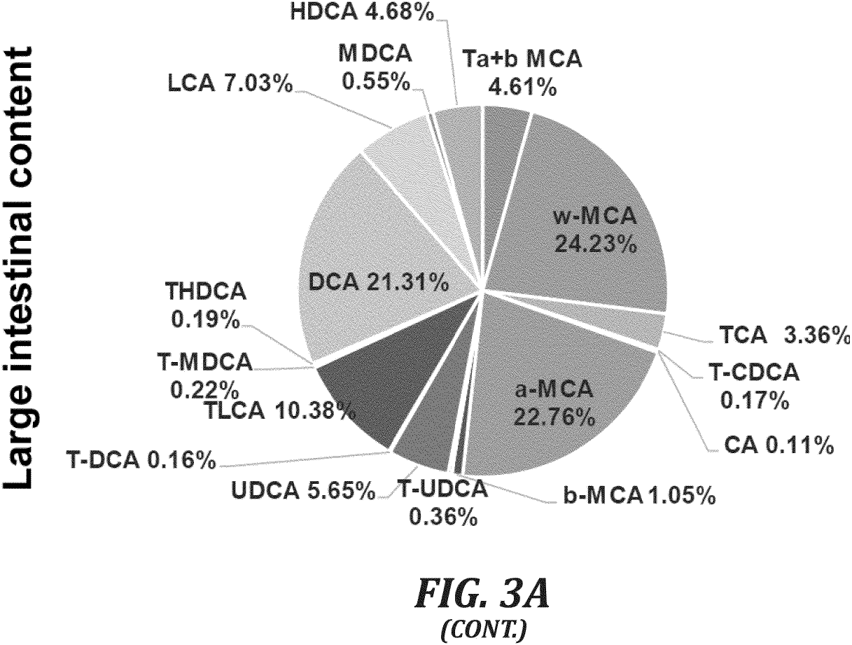

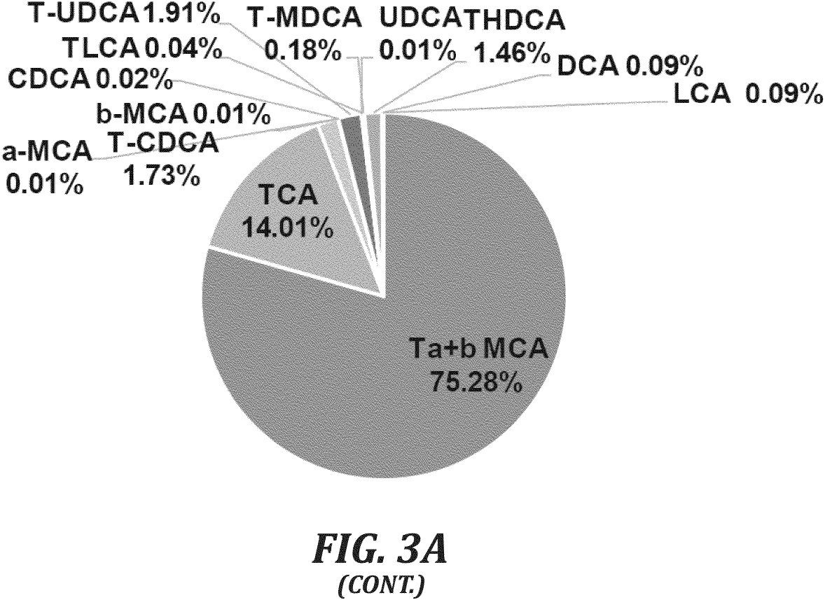

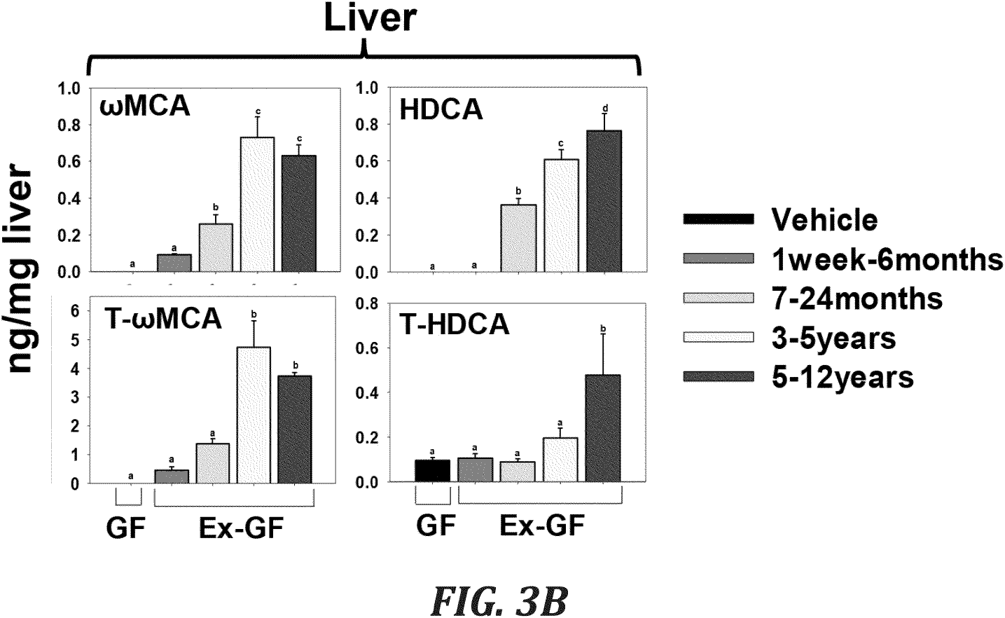

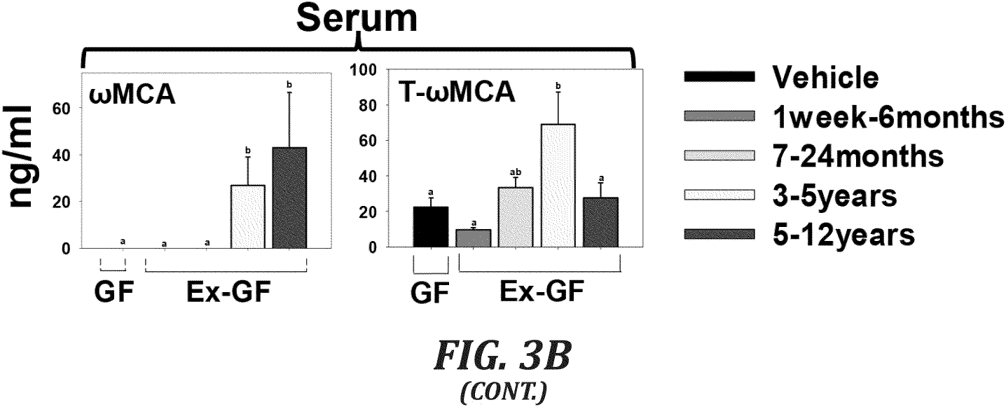

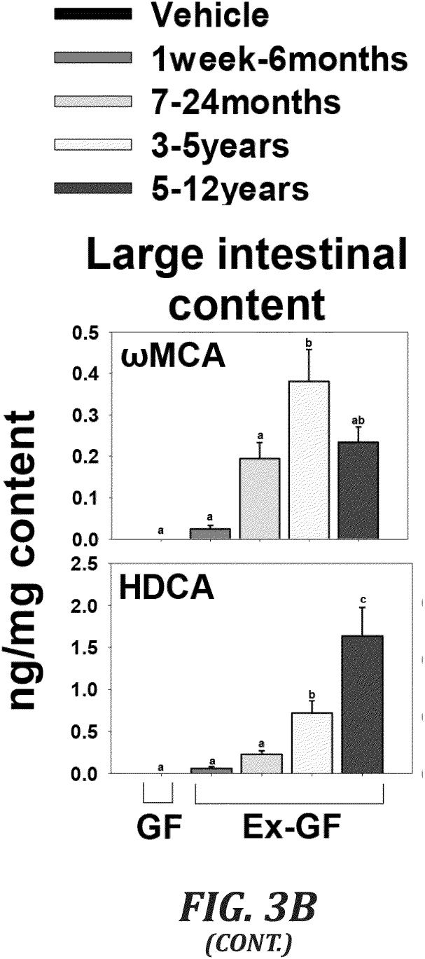

Gut microbiome-dependent production of ωMCA and the capacity of ωMCA regeneration by inoculating exGF mice with human feces. As shown in FIG. 3A, in serum of adult CV mice, the most abundant primary BAs detected are cholic acid (CA, 19.39%) and its taurine (T-) conjugated form (T-CA, 16.78%), followed by βMCA (15.56%), T-α/βMCA (3.43%), AND chenodeoxycholic acid (CDCA, 2.33%). Importantly, the most abundant microbially-derived secondary BA is ωMCA (17.84%), followed by deoxycholic acid (DCA, 9%) and T-DCA (2.89%). In contrast, LCA, which is known to activate PXR at by exogenous administration at high doses, occupy only 0.57% of the total serum pool and its conjugated form (T-LCA) occupies only 0.51%. Ursodeoxycholic acid (UDCA), which can be produced by both the host liver and microbiome in mice, occupies 1.85%, whereas the secondary BA T-HDCA (another product from βMCA), occupies 1.41%. Other BAs are minimally present in serum of CV mice. In serum of GF mice, ωMCA was completely absent along with the other major secondary BAs, whereas the most enriched BAs are T-α/βMCA (38.55%) and T-CA (18.11%). Similarly, in large intestinal content of CV mice, ωMCA was also the most abundant secondary BA (24.23%), followed by DCA (21.31%), T-LCA (10.38%), and LCA (7.03%). Proportions of most primary BAs in large intestinal content of CV mice are low due to extensive microbial metabolism of these substrates but αMCA (22.76%) is enriched, followed by T-α/βMCA (4.61%) and T-CA (3.36%). In large intestinal content of GF mice, the overwhelmingly enriched BA is T-α/βMCA (75.28%) followed by T-CA (14.01 %), and there is no ωMCA present. Together this observation indicates that the quantitatively most important microbial metabolism in mouse intestine is ωMCA production from its primary BA precursors, and this pathway is completely abolished in the absence of gut microbiome without any host metabolic compensations.

As shown in FIG. 3B, inoculating GF mice with human fecal microbiome of various ages increased ωMCA and its taurine-conjugated form in liver, large intestinal content, and serum in a human donor age-specific manner. Importantly, the colonized microbiome in exGF mice retained most of the features of the original human stool sample as evidenced by 16S rDNA sequencing confirmation (data not shown). In general, feces from 3 to about 5-year-old donors followed by 5 to about 12-year-old donors resulted the maximal increase in ωMCA in these bio-compartments. In addition, HDCA and T-HDCA were increased in liver whereas HDCA was increased in large intestinal content by human fecal microbiome transplant, with maximal increase observed of exGF mice inoculated by feces from 5 to about 12-year-old donors. To note, the taurine-conjugated forms of ωMCA and HDCA are minimal in large intestinal content, likely due to extensive deconjugation reactions by the inoculated bacteria. In summary, human fecal microbiome, when introduced to GF mice, can generate the mouse-specific ωMCA, suggesting that the species difference in ωMCA due to host production of the mouse-specific precursor βMCA, but not due to the difference in gut microbiome between mice and humans.

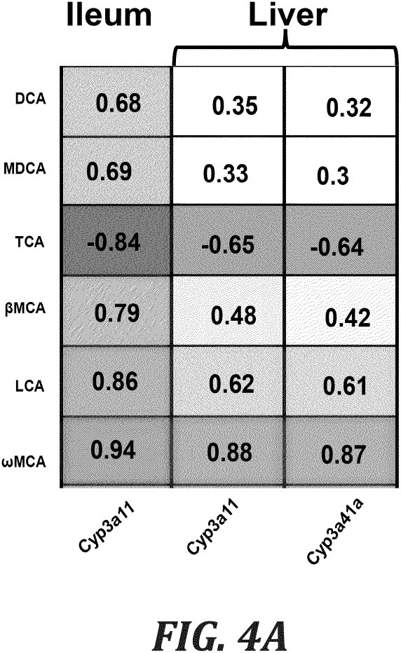

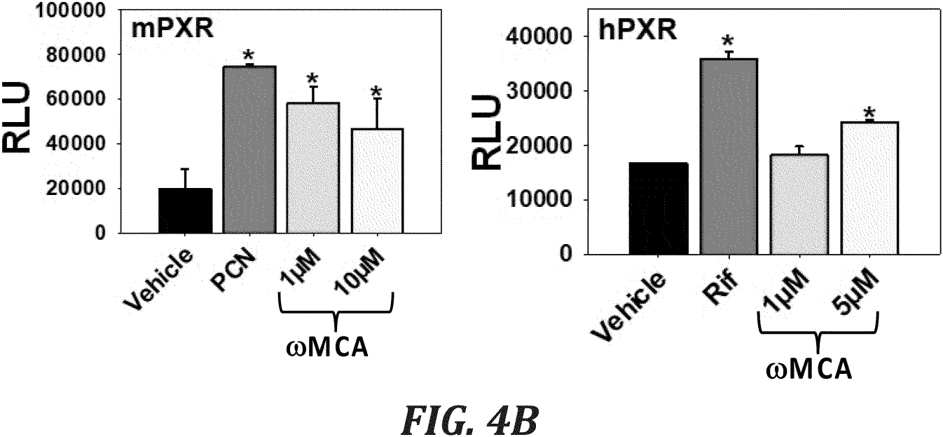

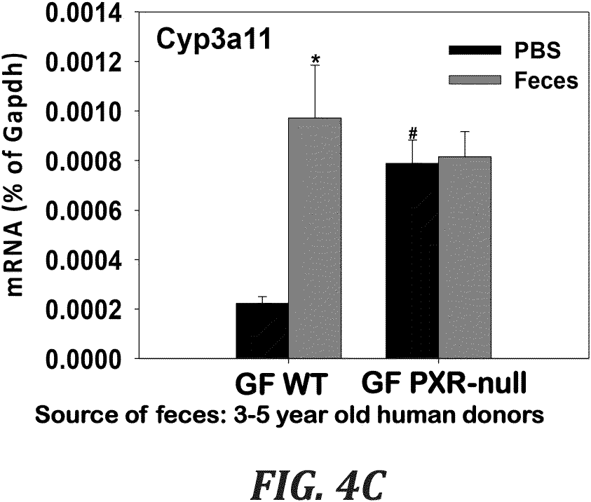

Building on the finding that PXR-signaling is attenuated in GF conditions (FIGS. 2A and 2B), and fecal microbiome transplant using human fecal microbiomes from various age-range groups increased the PXR-target genes Cyp3a and the most abundant secondary BA ωMCA (FIGS. 3A and 3B), Pearson’s correlation analysis was performed among all the differentially regulated intermediary metabolites in large intestinal content and the expression of PXR-target genes Cyp3a in ileum and liver (FIG. 4A).

The scope of the intermediary metabolites included in this study include all major SCFAs and their intermediate precursors (15), primary and secondary BAs (25) and various other aqueous metabolites from the metabolisms of amino acids, nucleotides, and carbohydrates. SCFAs and other aqueous metabolites were minimally associated with microbiome-dependent restoration of Cyp3a gene expression. Interestingly, among all the differentially regulated BAs, ωMCA in large intestinal content showed the strongest correlation with the human microbiome-mediated upregulation of Cyp3a11 mRNA in ileum (r = 0.94). ωMCA also positively associated with microbiome-dependent upregulation Cyp3a11and Cyp3a41a mRNAs in liver (r = 0.88 and 0.87, respectively) (FIG. 4A). Other secondary BAs (DCA, MDCA, LCA) and the primary BA βMCA (precursor ωMCA) positively associated Cyp3a11 in ileum but the r values were much weaker; and such association was further weakened in liver. T-CA, which the most abundant primary BA, showed negative associations with Cyp3a in both ileum and liver (FIG. 4A).

Luciferase reporter assay with mouse PXR (mPXR) plasmid showed that ωMCA activated mPXR in vitro at 1 µM and 10 µM concentrations. The prototypical mPXR ligand pregnenolone 16α-carbonitrile (PCN) was used as a positive control (FIG. 4B right panel). Luciferase reporter assay with human PXR (hPXR) plasmid showed that ωMCA also weakly activated hPXR in vitro at 5 µM. The prototypical hPXR ligand rifampicin (Rif) was used as a positive control (FIG. 4B left panel). The microbiome-dependent upregulation of Cyp3a11 was confirmed to be dependent on PXR, because human fecal microbiome from 3 to about 5-year-old donors, which resulted in the maximal restoration of Cyp3a in livers of GF WT mice, was not able to increase Cyp3a gene expression in livers of GF PXR-null mice (FIG. 4C).

In summary, these observations indicate that ωMCA is a novel PXR ligand for both mice and humans, because ωMCA is the most abundant microbially derived secondary BA in mice (FIG. 3A), the ωMCA-PXR signaling is likely both necessary and sufficient in maintaining the basal Cyp3a gene expression.

Pluripotent stem cell-derived human intestinal organoids (HIO) and tissue-derived duodenal enteroids (DE) have potential as physiologically relevant models for high-throughput validation experiments of FKK molecules in the human intestine. They self-organize into intestinal tissue-like structures that contain a higher percentage of non-enterocyte cell types than Caco-2, which are more indicative of native intestine phenotype. Both HIO and DE have been shown to express PXR, and PXR ligand FKK6 transcriptionally regulates known genes linked to PXR. For proof-of-concept, PXR knockout organoids were created using siRNA transfection. HIOs are generated from pluripotent stem cell line IISH1i-BM1 (Wicell) following the method described in Spence, J. R. et al. Directed differentiation of human pluripotent stem cells into intestinal tissue in vitro. Nature 470, 105-U120, doi:10.1038/nature09691 (2011), {Watson, 2014 #75} and DEs were generated from duodenal tissue explants. Stock HIOs and DEs were cultured in Intesticult™ in Matrigel beads to maintain a proliferative state. For experiments, HIOs and DEs were removed from Matrigel and cultured in suspension for 3 days in Advanced DMEM + 100 ng/ml EGF to promote differentiation and polarization to apical-out. For siRNA knockdown of PXR, HIOs and DEs were transfected with siRNA NR1I2 (PXR) with lipofectamine RNAiMax for 24 hours in Optimem. The system is utilized to characterize the biochemical and molecular interactions of ωMCA on PXR.

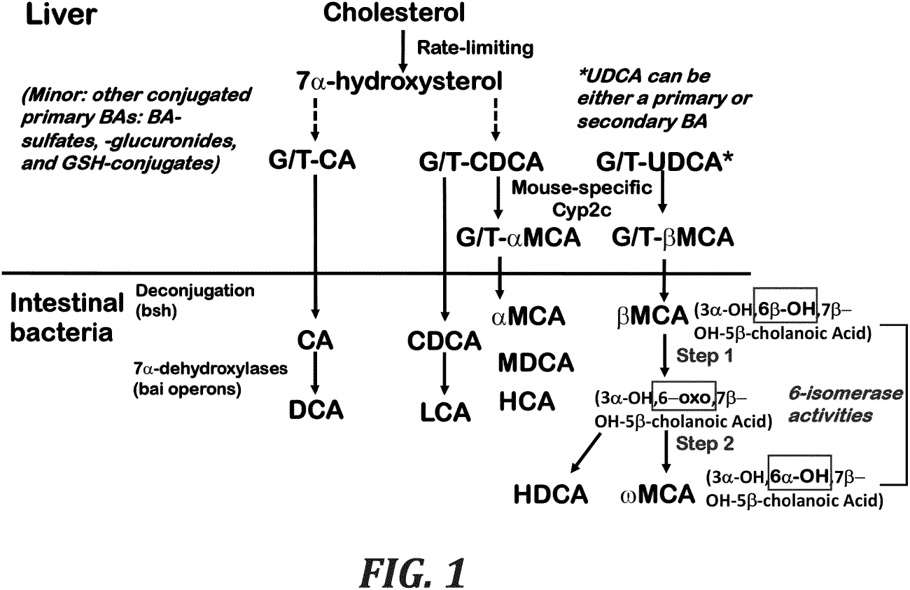

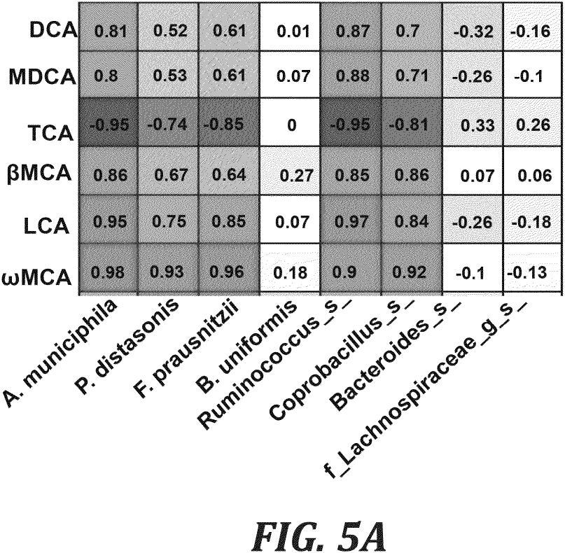

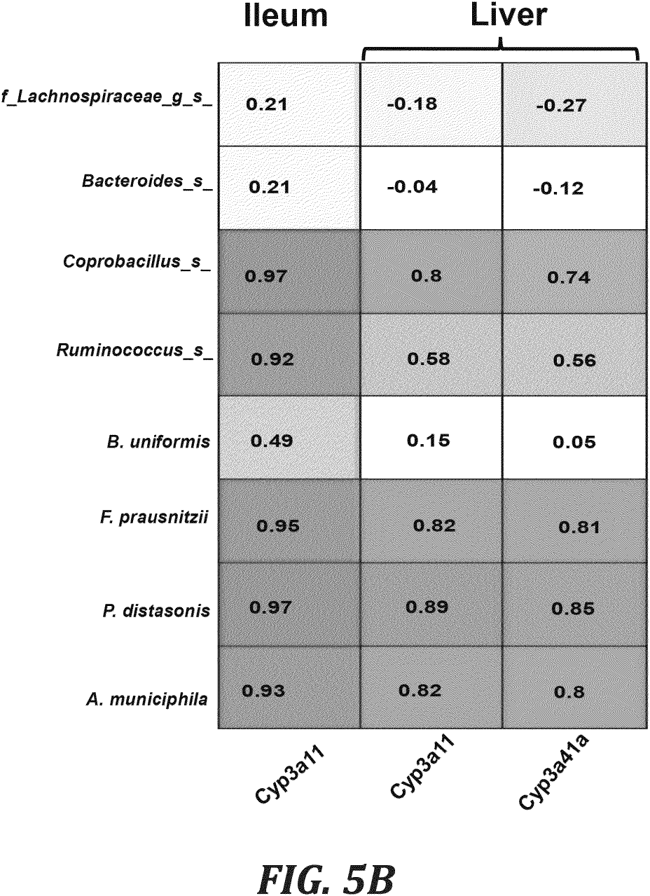

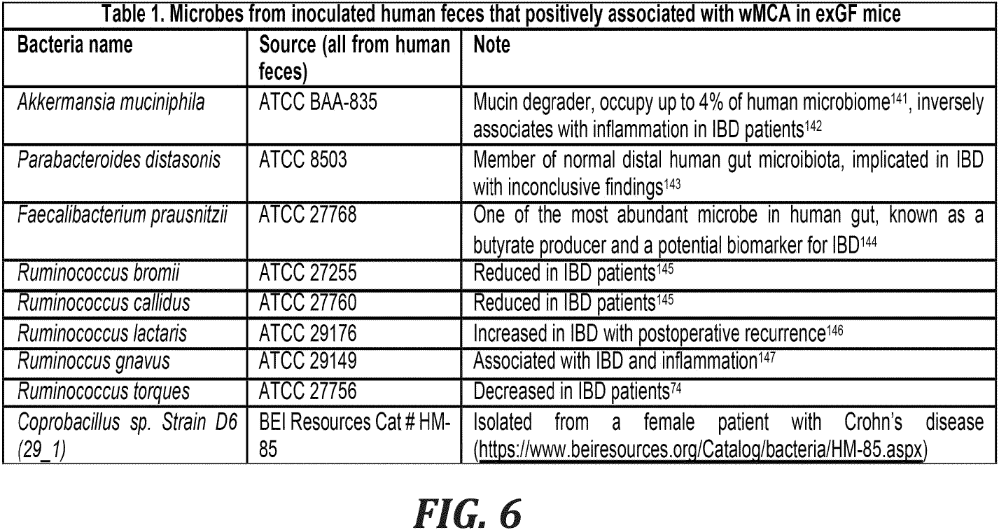

Novel microbes and microbial interactions in ωMCA production from its precursor βMCA were identified. t was demonstrated 40 years ago that E. lentum, which was later renamed as E. lenta, some atypical Fusobacterium sp. strain, and a strain of Clostridium group III, can convert βMCA to ωMCA (FIG. 1). These pioneer studies have markedly improved the understanding of the microbial 6 isomerase activities but have inherent limitations in the scope of the investigation due to lack of high throughput technologies to investigate all components and their interactions within the microbiome simultaneously. In addition, there are differences in the microbiome composition between rodents (mice and rats) and humans. Multi-omics integration identified 9 microbes (FIG. 6) from the inoculated human microbiome that are associated with ωMCA production and Cyp3a11 restoration (r > 0.9) in inoculated GF mice. As shown in FIG. 5A, in the study of GF mice inoculated with various human fecal microbiomes (detailed in FIG. 2B description) ωMCA levels in large intestinal content positively associated with A. muciniphila (r = 0.98), P. distasonis (r = 0.93), F. prausnitzii (r = 0.96), Ruminococcus (r = 0.9), and Coropbacillus (r = 0.92). Deep metagenomic shotgun sequencing further confirmed that the species positively associated with ωMCA in the Ruminococcus genus are: R. bromii, R. callidus, R. lactaris, R. sp. 5.1.39BFAA, R. gnavus, as well as R. obeum and R. torques (now grouped with the Blautia genus); whereas the species positively associated with ωMCA in the Coprobacillus genus is Coprobacillus sp. 29.1. These taxa described above were also positively associated with the human fecal microbiome-mediated restoration of Cyp3a in ileum (r = 0.92 ~ 0.97) and liver (r = 0.56 ~ 0.85) (FIG. 5B). βMCA is microbially transformed into ωMCA via 6-isomerase or hyodeoxycholic acid (HDCA), and the ratio of ωMCA/HDCA is profoundly increased by autoclaved lactose-containing starch diet but suppressed by a high fiber diet.

Building on these findings, the 6-isomerase activity were tested in these microbes cultured individually or in combinations with deuterium (²H) labeled βMCA, and conducting isotope-tracing in feces, intestine, and liver of mice fed with ¹³C labeled βMCA to examine the microbial combinations contributing to ωMCA vs. HDCA roduction in mice fed with various diets. The 9-microbe culture/co-culture or distinct colonies from the stool isolates have 6-isomerase activity for βMCA ➔ ωMCA conversion. This reaction occurs in two steps (6βOH ➔ 6-oxo ➔ 6αOH). The microbes/microbial combination in inoculated GF mice are expected to convert βMCA to ωMCA in vivo, corresponding to increased PXR- signaling in host organs such as liver and/or intestine. It is important to note that although βMCA and ωMCA are both rat- and mouse-specific BAs, as described herein it has been demonstrated that the human fecal microbiome can generate ωMCA if the precursor βMCA is present (as in the GF mice) (FIGS. 3A and 3B). Considering that βMCA has already demonstrated great therapeutic potential to treat metabolic disorders through antagonizing FXR and obesity is prevalent among IBD patients (15-40%), a combination of βMCA + ωMCA-producing probiotics with to further enhance the endogenous 6-isomerase activities will bring even greater therapeutic values in patients with multiple complex diseases (e.g., IBD + obesity) through a “FXR ➔ PXR” 2-hit strategy.

The present disclosure demonstrates that the most abundant secondary BA ωMCA in mice is a novel ligand of the host drug receptor PXR of both mouse and human origins. As also escribed herein, the human microbiome can generate the mouse specific ωMCA in vivo when the substrate βMCA is present.

As noted above, the present disclosure characterizes the biochemical and molecular interactions of omega muricholic acid (ωMCA) on pregnane X receptor (PXR), identifies additional novel microbes and microbial interactions in ωMCA production from its precursor βMCA, and tests the effect of ωMCA in several murine models of inflammatory bowel disease (IBD) and nonalcoholic steatohepatitis (NASH).

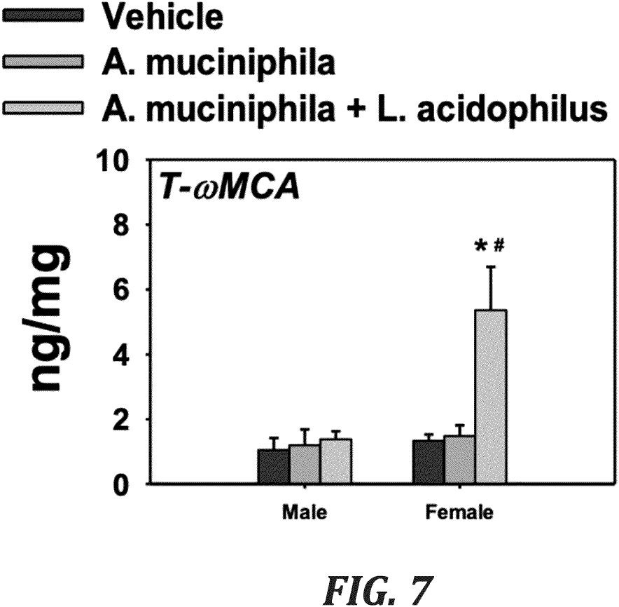

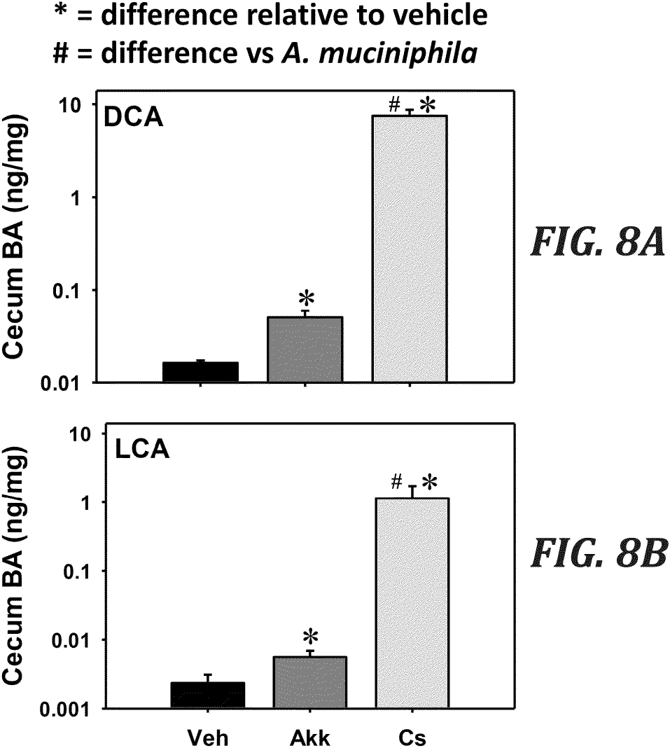

ωMCA can be produced by human fecal microbiome in inoculated exGF mice, and it is a novel PXR activator of both mouse and human origins. Given the high tissue abundance of ωMCA in vivo, ωMCA is likely a main contributor of the constitutive PXR signaling in liver and intestine and may be harnessed therapeutically to treated liver and/or GI diseases. In support of the therapeutic effectiveness of ωMCA, (a) in vivo experiments were conducted using inoculated germ-free mice to identify mechanistic novel microbial interactions ( A. muciniphila + L. acidophilus) in ωMCA production in a sex-dependent manner (see FIG. 7), (b) ex vivo experiments were conducted using isolated mouse cecum showing that A. muciniphila can produce moderate levels of other microbially derived secondary bile acids, namely deoxycholic acid and lithocholic acid, which can also activate PXR (see FIGS. 8A and 8B), and (c) docking experiments were conducted investigating the candidates of ωMCA mimicry in PXR activation (see Table 1). Together these additional data demonstrate the development of ωMCA as a target towards PXR.

A. muciniphila in combination with L. acidophilus results in increased intestinal taurine-conjugated wMCA in inoculated female ex-germ-free mice. A. muciniphila and L. acidophilus were cultured in vivo using an anaerobic chamber (freeze-dried strains were purchased from ATCC). As a quality control to ensure the rigor of our experiments, a time-course study was conducted using inoculated ex-germ-free mice and quantified the specific microbes in fresh fecal samples using quantitative qPCR and confirmed that the inoculated microbes successfully colonized in mouse intestine and their abundance was stable over time.

As shown in FIG. 7, interestingly, ωMCA in its taurine-conjugated form (T-ωMCA which is the majority of its presence in liver and circulation) was not altered in A. muciniphila singly inoculated group; however, A. muciniphila in combination with L. acidophilus (which does not have the capacity in ωMCA production) resulted in a marked increase in T-ωMCA production in females.

In addition to ωMCA production, ex vivo experiments using isolated cecum from inoculated ex-germ-free mice showed that A. muciniphila can produce moderate levels of other microbially derived secondary bile acids, namely deoxycholic acid (DCA) and lithocholic acid (LCA), which can also activate PXR (FIGS. 8A and 8B).

The data at the mechanistic level establishes a novel microbial interaction in ωMCA production and suggest that A. muciniphila requires partner(s) from the microbial community to activate the host PXR receptor. The sex-specificity is likely due to male-specific microbial deconjugation of T-ωMCA and/or additional metabolic pathways in T-ωMCA metabolism. It is also possible that these microbes indirectly impact the host hepatic bio-synthesis of primary bile acids through remote sensing mechanisms using certain microbial metabolites that they produce.

Docking experiment and luciferase assays showing candidate bile acid metabolites that are PXR activators. ωMCA mimicries were explored by looking at LCA and 3-keto-LCA (dehydrolithocholic acid) that effectively activate human PXR. Therefore, ωMCA derivatives were examined and found that among various bile acid metabolites, murideoxycholic acid docking experiments showed high similarity between the two compounds that are known hPXR activators, in particular, lithocholic acid. The results are summarized in Table 1.

TABLE 1

Agonist and Antagonist Scores for Select Bile Acids

| Compound | Agonist score | Antagonist score |
|---|---|---|
| omega muricholic acid | 58.52 | 44.43 |
| murideoxycholic acid | 60.18 | 47.24 |
| lithocholic acid | 54.71 | 47.28 |
| dehydroxylithocholic acid | 55.01 | 45.46 |

Structurally similar bile acids are not effective PXR activators. As described herein, the effectiveness of ωMCA as a PXR activator has been demonstrated. However, structurally similar bile acids (e.g., murideoxycholic acid (MDCA)) do not have the same advantageous PXR activity.

The chemical structure of MDCA is as follows:

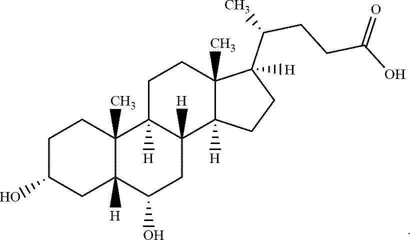

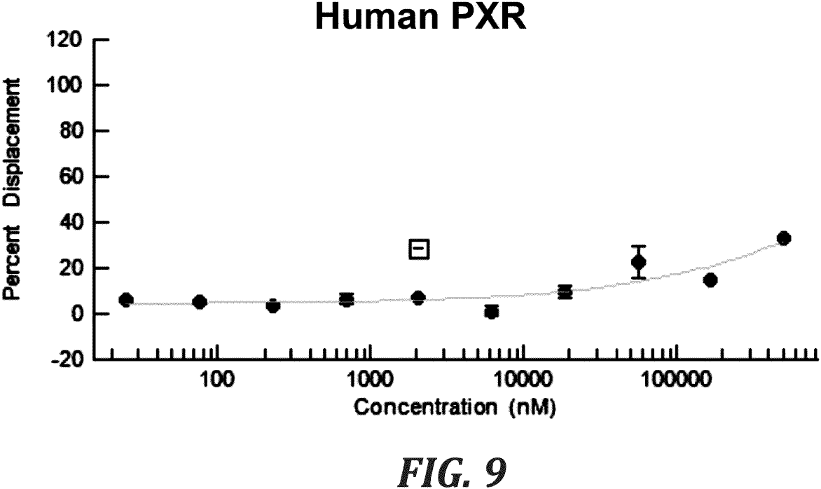

Percent displacement of reporter from human PXR as a function of MDCA concentration was evaluated and the results are shown in FIG. 9. In this evaluation, the binding of murideoxycholic acid (MDCA), a bile acid that shares a structural similarity to omega muricholic acid (ωMCA), was evaluated.

A reporter assay was performed using LS intestinal cells transfected with human PXR + reporter. Incubation time was 24 h and a dose-response curve was plotted. Results were confirmed using four different cells passages. Although MDCA has binding affinity toward PXR that is predicted by bioinformatics analysis to be similar to ωMCA, MDCA exhibited no binding affinity towards PXR in experimental settings. MDCA is totally inactive up to the maximum tested concentration. This is an unexpected result based on the effective binding exhibited by structurally similar ωMCA.

### Definitions

The human pregnane X receptor (PXR, NR1I2, UniProtKB ID: O75469) is a nuclear receptor that binds and is usually activated by a variety of xenobiotics and endogenous chemicals. PXR is evolutionarily conserved and the mouse PXR (Nrli2, UniProtKB ID: 054915) shares many similar functions with the human PXR. For example, upon activation, PXR of both species is translocated from cytosol into nucleus and binds to the response elements in DNA, and this usually leads to trans-activation of many genes involved in the metabolism and transport of drugs and other chemicals (PMID: 11248086; PMID: 27709013).

The chemical structure of ω-muricholic acid is as follows:

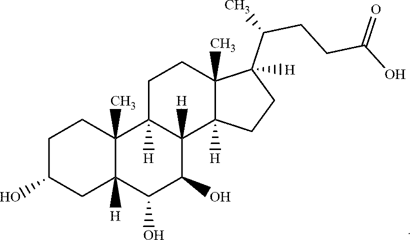

The chemical structure of P-muricholic acid is as follows:

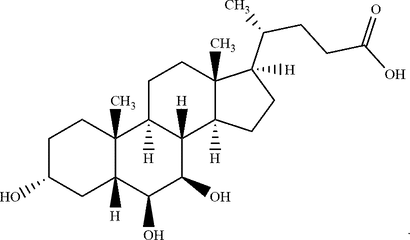

As used herein, “salts” include derivatives of an active agent, wherein the active agent is modified by making acid or base addition salts thereof. Examples of pharmaceutically acceptable salts include, but are not limited to, mineral or organic acid addition salts of basic residues such as amines; alkali or organic addition salts of acidic residues; and the like, or a combination comprising one or more of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, or a combination comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC— (CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; or a combination comprising one or more of the foregoing salts.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The terms “therapeutic agent”, “active agent”, “drug”, and “active pharmaceutical ingredient” are used interchangeably herein.

As used herein, a “composition” refers to a collection of materials containing the specified components. One or more dosage forms may constitute a composition, so long as those dosage forms are associated and designed for use together.

As used herein, a “pharmaceutical composition” refers to a formulation of a compound/combination of compounds of the disclosure, and a medium generally accepted in the art for the delivery of the biologically active compound to mammals, e.g., humans. Such a medium includes all pharmaceutically acceptable carriers, diluents, or excipients therefor. The pharmaceutical composition may be in various dosage forms or contain one or more unit-dose formulations. The pharmaceutical composition can provide stability over the useful life of the composition, for example, for a period of several months. The period of stability can vary depending on the intended use of the composition.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:
preventing the disease; for example, preventing a disease, condition or disorder in an individual who may be predisposed to the disease, condition or disorder but does not yet experience or display the pathology or symptomatology of the disease;
inhibiting the disease; for example, inhibiting a disease, condition or disorder in an individual who is experiencing or displaying the pathology or symptomatology of the disease, condition, or disorder; and
ameliorating the disease; for example, ameliorating a disease, condition or disorder in an individual who is experiencing or displaying the pathology or symptomatology of the disease, condition, or disorder (i.e., reversing the pathology and/or symptomatology) such as decreasing the severity of disease.

As used herein, “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. “Administering” can also include prescribing or filling a prescription for a dosage form comprising a particular compound/combination of compounds, as well as providing directions to carry out a method involving a particular compound/combination of compounds or a dosage form comprising the compound/combination of compounds.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

The term “about” in reference to a numerical value refers to the range of values somewhat less or greater than the stated value, as understood by one of skill in the art. As used herein, the term “about” refers to ± 5% of the stated value.

At various places in the present specification, groups or ranges are described. It is specifically intended that the disclosure include each and every individual sub-combination of the members of such groups and ranges.

Where a numerical range is disclosed herein, such a range is continuous, inclusive of both the minimum and maximum values of the range, as well as every value between such minimum and maximum values. Still further, where a range refers to integers, every integer between the minimum and maximum values of such range is included. In addition, where multiple ranges are provided to describe a feature or characteristic, such ranges can be combined. That is to say that, unless otherwise indicated, all ranges disclosed herein are to be understood to encompass any and all subranges subsumed therein. For example, a stated range of from “1 to 10” should be considered to include 1 and 10, and any and all subranges between the minimum value of 1 and the maximum value of 10. Exemplary subranges of the range “1 to 10” include, but are not limited to, e.g., 1 to 6.1, 3.5 to 7.8, and 5.5 to 10.

While illustrative embodiments have been illustrated and described, it will be appreciated that various changes can be made therein without departing from the spirit and scope of the invention.

1. A method of activating a pregnane X receptor (PXR) in a subject, comprising administering to a subject in need thereof a therapeutically effective amount of ω-muricholic acid, or a pharmaceutically acceptable salt thereof.

2. The method of claim 1, wherein the ω-muricholic acid is generated in vivo from P-muricholic acid, or a pharmaceutically acceptable salt thereof, and a bacterium capable of converting β-muricholic acid, or a pharmaceutically acceptable salt thereof, to ω-muricholic acid, or a pharmaceutically acceptable salt thereof.

3. The method of claim 2, wherein the bacterium is selected from the group consisting of Akkermansia muciniphila, Parabacteroides distasonis, Faecalibacterium prausnitzii, Ruminococcus bromii, Ruminococcus callidus, Ruminococcus lactaris, Ruminoccus gnavus, Ruminococcus torques, Coprobacillus sp. Strain D6, and combinations thereof.

4. The method of claim 1 further comprising administering to the subject P-muricholic acid, or a pharmaceutically acceptable salt thereof, concurrently with the ω-muricholic acid, or a pharmaceutically acceptable salt thereof.

5. The method of claim 4 further comprising administering to the subject a bacterium capable of converting β-muricholic acid, or a pharmaceutically acceptable salt thereof, to ω-muricholic acid, or a pharmaceutically acceptable salt thereof.

6. The method of claim 5, wherein the bacterium is selected from the group consisting of Akkermansia muciniphila, Parabacteroides distasonis, Faecalibacterium prausnitzii, Ruminococcus bromii, Ruminococcus callidus, Ruminococcus lactaris, Ruminoccus gnavus, Ruminococcus torques, Coprobacillus sp. Strain D6, and combinations thereof.

7. The method of claim 1, wherein activating PXR comprises contacting PXR with ω-muricholic acid, or a pharmaceutically acceptable salt thereof.

8. The method of claim 7, comprising selectively activating PXR in the intestines of the subject.

9. The method of claim 1, wherein administrating comprises oral or rectal administration.

10. The method of claim 1, wherein ω-muricbolic acid, or a pharmaceutically acceptable salt thereof, is administered at a ω-muricbolic acid-equivalent dose compatible with the activation profile of PXR in the intestines and/or other organs.

11. The method of claim 1, wherein the ω-muricbolic acid, or a pharmaceutically acceptable salt thereof, comprises a dosage form selected from a solid dosage form, a liquid dosage form, or a suspension.

12. The method of claim 1, wherein the subject has a condition selected from metabolic syndrome, obesity, inflammatory bowel disease, Crohn’s disease, liver disease, or a combination thereof.

13. A method of treating metabolic syndrome, obesity, inflammatory bowel disease, Crohn’s disease, liver disease, or increasing CYP3A4 gene and/or protein expression in a subject, comprising administering to a subject in need thereof a therapeutically effective amount of co-muricholic acid, or a pharmaceutically acceptable salt thereof.

14. The method of claim 13 further comprising treating a comorbid liver disease in the subject.

15. The method of claim 14, wherein the comorbid liver disease is selected from nonalcoholic fatty liver disease, nonalcoholic steatohepatitis, or a combination thereof.

16. The method of claim 13 further comprising administering to the subject a therapeutically effective amount of P-muricholic acid, or a pharmaceutically acceptable salt thereof, and a bacterium capable of converting β-muricholic acid, or a pharmaceutically acceptable salt thereof, to co-muricholic acid, or a pharmaceutically acceptable salt thereof.

17. The method of claim 16, wherein the bacterium comprises Akkermansia muciniphila, Parabacteroides distasonis, Faecalibacteriumprausnitzii, Ruminococcus bromii, Ruminococcus callidus, Ruminococcus lactaris, Ruminoccus gnavus, Ruminococcus torques, Coprobacillus sp. Strain D6, or any combination thereof.

18. A method of treating metabolic syndrome, obesity, inflammatory bowel disease, Crohn’s disease, liver disease, or increasing CYP3A4 gene and/or protein expression in a subject, comprising administering to a subject in need thereof a therapeutically effective amount of P-muricholic acid and a bacterium capable of converting β-muricholic acid to ω-muricbolic acid.

19. The method of claim 1, wherein the subject is a human.

20. The method of claim 13, wherein the subject is a human.
